(19) United States
(12) Patent Application Publication
(10) Pub. No.: US 2026/0199149 A1
(43) Pub. Date: Jul. 16, 2026

(54) ABSORBENT SHEET

(71) Applicant: Unicharm Corporation, Shikokuchuo-shi, Ehime (JP)

(72) Inventors: Yumi ETOH, Kanonji-shi, Kagawa (JP); Mami YAMASHITA, Kanonji-shi, Kagawa (JP); Akihiro KIMURA, Kanonji-shi, Kagawa (JP)

(21) Appl. No.: 19/139,152

(22) PCT Filed: Dec. 14, 2023
(86) PCT No.: PCT/JP2023/044886

§ 371 Date: Jun. 13, 2025

(30) Foreign Application Priority Data
Dec. 20, 2022 (JP) 2022-203122

(51) Int. Cl. A61F 13/533; A61F 13/15; A61F 13/53; A61F 13/539

(52) U.S. Cl. CPC A61F 13/533; A61F 2013/15373; A61F 2013/530007; A61F 2013/530496; A61F 2013/53908

(57) ABSTRACT

This absorbent sheet (1) has: an absorbent core (10) provided with a liquid-absorbing fiber and a superabsorbent polymer; a liquid-permeable front sheet (11); and a back sheet (12). The maximum thickness of the absorbent sheet (1) is 2.0 mm or less, and both the average value of longitudinal bending rigidity B of the absorbent sheet (1) according to KES and the average value of lateral bending rigidity B of the absorbent sheet (1) according to KES are 0.5 gf·cm²/cm or more.

### FIELD

The present disclosure relates to an absorbent sheet.

### BACKGROUND

As absorbent sheets, pet absorbent sheets used for pet excrement disposal have been known. PTL 1 discloses a pet absorbent sheet in which an absorbent body is interposed between a liquid-permeable top sheet and liquid-impermeable back sheet. PTL 1 also discloses that, in order to prevent a tear of the back sheet caused by friction with a superabsorbent resin even in the back sheet having a low-basis-weight, the absorbent body has a two-layered structure constituted by an upper-layer-absorbent-body having the superabsorbent resin and a lower-layer-absorbent-body without the superabsorbent resin.

### CITATION LIST

### Patent Literature

[PTL 1] Patent No. 6148912

### SUMMARY

### Technical Problem

For example, in the case where the absorbent sheet with low rigidity is used for pets, the absorbent sheet is likely to be twisted (crumpled) when a dog or cat moves around on the absorbent sheet. This reduces an absorption area of the absorbent sheet, which makes excreted fluid likely to leak from the absorbent sheet.

However, as in the case of PTL 1 in which the absorbent sheet has the two-layered structure formed by the material (lower-layer-absorbent-body) being added to the absorbent body to increase the rigidity of the absorbent sheet, the thickness of the absorbent sheet increases. This may cause a height difference between the absorbent sheet and a placement surface of the absorbent sheet so that dog's or cat's legs are likely to be caught on such a height difference, which makes the absorbent sheet likely to curl. When the absorbent sheet curls, the absorption area is covered with a curled portion of the absorbent sheet, thereby making the excreted fluid likely to leak from the absorbent sheet.

PTL 1 does not disclose a state after the absorbent sheet absorbs the excreted fluid, but the absorbent sheet after absorption of the excreted fluid bulges and increases in thickness. In the case where the absorbent sheet after absorption varies greatly in bulge and has significant unevenness on the surface thereof, the dog's or cat's legs are likely to be caught on such an uneven surface. This causes the twisting or curling of the absorbent sheet. Then, the absorption area of the absorbent sheet is reduced, which makes the excreted fluid more likely to leak from the absorbent sheet at a time of subsequent excretion. On the other hand, reduction in the amount of absorption (water retention) of the absorbent sheet can reduce the bulge of and the surface unevenness (variation) of the absorbent sheet after absorption. However, in the case where the amount of absorption of the absorbent sheet is low, the excreted fluid is likely to leak from the absorbent sheet.

The present disclosure was achieved in light of conventional problems such as that described above, and an aspect of the present disclosure is to provide an absorbent sheet in which reduction in an absorption area due to twisting and curling of the absorbent sheet is suppressed and leakage is less likely to occur.

### Solution to Problem

A main aspect of the present disclosure for achieving the above-described aspect is an absorbent sheet having longitudinal direction, a lateral direction, and a thickness direction that are orthogonal to each other, the absorbent sheet including: an absorbent core having liquid-absorbent fiber and superabsorbent polymer; a top-face sheet disposed on a top-face side in the thickness direction with respect to the absorbent core, the top-face sheet being liquid permeable; and a back-face sheet disposed on a back-face side in the thickness direction with respect to the absorbent core, a maximum thickness of the absorbent sheet being 2.0 mm or less, an average value of a bending rigidity B of the absorbent sheet in the longitudinal direction according to a KES method being 0.5 gf·cm²/cm or more, an average value of a bending rigidity B of the absorbent sheet in the lateral direction according to the KES method being 0.5 gf·cm²/cm or more. Features of the present disclosure other than the above will become clear by reading the description of the present specification with reference to the accompanying drawings.

### Advantageous Effects of Invention

According to the present disclosure, an absorbent sheet in which reduction in an absorption area due to twisting and curling of the absorbent sheet is suppressed and leakage is less likely to occur, can be provided.

### BRIEF DESCRIPTION OF THE DRAWINGS

FIG. 1 is a plan view of an absorbent sheet 1 as viewed in the thickness direction.

FIG. 2A and FIG. 2B are each a side view of the absorbent sheet 1, taken along the thickness direction.

FIG. 3 is an illustrative diagram of a method of manufacturing an absorbent-body continuous body 2′.

FIG. 4 is an illustrative diagram of a method of manufacturing the absorbent sheet 1.

FIG. 5A and FIG. 5B are each an illustrative diagram of a method of measuring the bending rigidity B.

FIG. 6 is diagram showing measurement results of absorbent sheets 1 of the present embodiments (Examples A and B) and Conventional Products A to E.

FIG. 7A is an illustrative diagram of divided regions R1 to R16, and FIG. 7B is an illustrative diagram of a drop test of artificial urine.

FIG. 8 is a diagram showing variations in thickness and results of the drop test.

FIG. 9 is a diagram illustrating adhesives 60 that join a back-face sheet 12 and a core-wrapping sheet 14 together.

FIG. 10A is a diagram illustrating a method of measuring variation in weight of the absorbent sheet 1, and FIG. 10B is a diagram showing measurement results of variation in weight of the absorbent sheet 1.

### DESCRIPTION OF EMBODIMENTS

At least the following matters will become clear with description of this specification and attached drawings.

### Aspect 1

An absorbent sheet having a longitudinal direction, a lateral direction, and a thickness direction that are orthogonal to each other, the absorbent sheet including: an absorbent core having liquid-absorbent fiber and superabsorbent polymer; a top-face sheet disposed on a top-face side in the thickness direction with respect to the absorbent core, the top-face sheet being liquid permeable; and a back-face sheet disposed on a back-face side in the thickness direction with respect to the absorbent core, a maximum thickness of the absorbent sheet being 2.0 mm less, an average value of a bending rigidity B of the absorbent sheet in the longitudinal direction according to a KES method being 0.5 gf·cm²/cm or more, an average value of a bending rigidity B of the absorbent sheet in the lateral direction according to the KES method being 0.5 gf·cm²/cm or more.

According to the absorbent sheet of Aspect 1, the absorbent sheet is less likely to be twisted (less likely to be crumpled) even when forces in various directions are applied, as compared with the case where the average value of the bending rigidity B in at least one of the longitudinal direction and the lateral direction is less than 0.5 gf·cm²/cm. In addition, it can be suppressed that the absorbent sheet curls due to catching on the height difference between the absorbent sheet and the placement surface of the absorbent sheet, as compared with the maximum thickness is greater than 2.0 mm. Thus, reduction in the absorption area due to the twisting and curling of the absorbent sheet can be suppressed, which can suppress the leakage from the absorbent sheet.

### Aspect 2

The absorbent sheet according to Aspect 1, wherein, the absorbent sheet is divided into four portions in the longitudinal direction and into four portions in the lateral direction, the divided portions in the absorbent sheet are defined as divided regions, in the absorbent sheet subjected to a drop test, the drop test being performed by dropping artificial urine at a predetermined position on the absorbent sheet, the divided regions where the artificial urine diffuses are defined as diffusion divided regions, the absorbent sheet before the drop test has a pre-absorption thickness that is a thickness c portion of each of the diffusion divided regions where the absorbent core is present, the absorbent sheet after the drop test has a post-absorption thickness that is a thickness of a portion of each of the diffusion divided regions where the absorbent core is present, and a CV value that indicates variation in a value obtained by subtracting the pre-absorption thickness from the post-absorption thickness in each of the diffusion divided regions, is 30% or less.

According to the absorbent sheet of Aspect 2, variation in the bulge value is smaller as compared with the case where the CV value of the value (bulge value) obtained by subtracting the pre-absorption thickness from the post-absorption thickness is greater than 30%. This can reduce unevenness on the sheet surface after absorption. This can suppress folding of the sheet due to the thickness difference, and catching on protruded portions. Thus, the twisting of the absorbent sheet after absorption can be suppressed. Thus, even when the absorbent sheet repeatedly absorbs the excreted fluid and the like, the absorption area can be ensured to suppress the leakage from the absorbent sheet.

### Aspect 3

The absorbent sheet according to Aspect 2, wherein, a maximum value obtained by subtracting the pre-absorption thickness from the post-absorption thickness in each of the diffusion divided regions is 1 mm more.

According to the absorbent sheet of Aspect 3, as compared with the case where the maximum value of the value (bulge value) obtained by subtracting the pre-absorption thickness from the post-absorption thickness is less than 1 mm, the absorbent sheet bulges by reliably absorbing and retaining the excreted fluid and the like. This can suppress the leakage from the absorbent sheet.

### Aspect 4

An absorbent sheet having a longitudinal direction, lateral direction, and a thickness direction that are orthogonal to each other, the absorbent sheet including: an absorbent core having liquid-absorbent fiber and superabsorbent polymer; a top-face sheet disposed on a top-face side in the thickness direction with respect to the absorbent core, the top-face sheet being liquid permeable; and, a back-face sheet disposed on a back-face side in the thickness direction with respect to the absorbent core, the absorbent sheet being divided into four portions in the longitudinal direction and into four portions in the lateral direction, the divided portions in the absorbent sheet being defined as divided regions, in the absorbent sheet subjected to a drop test, the drop test being performed by dropping artificial urine at a predetermined position on the absorbent sheet, the divided regions where the artificial urine diffuses being defined as diffusion divided regions, the absorbent sheet before the drop test having a pre-absorption thickness that is a thickness of a portion of each of the diffusion divided regions where the absorbent core is present, the absorbent sheet after the drop test having a post-absorption thickness that is a thickness of a portion of each of the diffusion divided regions where the absorbent core is present, a CV value that indicates variation in a value obtained by subtracting the pre-absorption thickness from the post-absorption thickness in each of the diffusion divided regions, being 30% or less, a maximum value obtained by subtracting the pre-absorption thickness from the post-absorption thickness in each of the diffusion divided regions, being 1 mm or more.

According to the absorbent sheet of Aspect 4, variation in the bulge value is smaller as compared with the case where the CV value of the value (bulge value) obtained by subtracting the pre-absorption thickness from the post-absorption thickness is greater than 30%. This can reduce unevenness on the sheet surface after absorption. This can suppress folding of the sheet due to the thickness difference, and catching on protruded portions. Thus, the twisting of the absorbent sheet after absorption can be suppressed. Thus, even when the absorbent sheet repeatedly absorbs the excreted fluid and the like, the absorption area can be ensured to suppress the leakage from the absorbent sheet. Furthermore, as compared with the case where the maximum value of the value (bulge value) obtained by subtracting the pre-absorption thickness from the post-absorption thickness is less than 1 mm, the absorbent sheet bulges by reliably absorbing and retaining the excreted fluid and the like. This can suppress the leakage from the absorbent sheet.

### Aspect 5

The absorbent sheet according to any one of Aspects 2 to 4, wherein, a maximum value obtained by subtracting the pre-absorption thickness from the post-absorption thickness in each of the diffusion divided regions, being 2 mm or less.

According to the absorbent sheet of Aspect 5, as compared with the maximum value of the value (bulge value) obtained by subtracting the pre-absorption thickness from the post-absorption thickness is greater than 2 mm, the localized bulging does not occur in the absorbent sheet. This can reduce the thickness difference between the diffusion divided regions and other regions (specifically, portions where the excreted fluid has not diffused). This can suppress folding of the sheet due to the thickness difference, and catching on protruded portions. Thus, the twisting of the absorbent sheet after absorption can be suppressed to ensure the absorption area.

### Aspect 6

The absorbent sheet according to any one of Aspects 1 to 5, wherein, the absorbent sheet is divided into four portions in the longitudinal direction and into four portions in the lateral direction, the divided portions in the absorbent sheet being defined as divided regions, and, in the absorbent sheet before absorption, a CV value that indicates variation in a thickness of a portion of each of the divided regions where the absorbent core is present is 10% or less.

According to the absorbent sheet of Aspect 6, unevenness on the sheet surface before absorption is reduced, as compared with the case where the CV value of the thickness before absorption is greater than 10%. This can suppress folding of the sheet due to the thickness difference, and catching on protruded portions. Thus, the twisting of the absorbent sheet before absorption can be suppressed to ensure the absorption area.

### Aspect 7

The absorbent sheet according to any one of Aspects 1 to 6, wherein, a core-wrapping sheet is disposed between the absorbent core and at least one of the top-face sheet and the back-face sheet, and, pulp fiber included in the liquid-absorbent fiber is hydrogen-bonded with cellulosic fiber included in the core-wrapping sheet.

According to the absorbent sheet of Aspect 7, the absorbent core and the core-wrapping sheet are firmly joined together. This can increase the rigidity of the absorbent sheet. In addition, shifting of the materials (such as the core-wrapping sheet) can be suppressed inside the absorbent sheet, thereby suppressing the twisting of the absorbent sheet.

### Aspect 8

The absorbent sheet according to any one of Aspects 1 to 7, wherein, a back surface of an absorbent body including the absorbent core is joined onto the back-face sheet by an adhesive, the adhesive is provided continuously in the longitudinal direction over an entire longitudinal range of the back surface of the absorbent body, and, the adhesive is provided continuously in the lateral direction over an entire lateral range of the back surface of the absorbent body.

According to the absorbent sheet of Aspect 8, the absorbent body and the back-face sheet are firmly joined together, which can improve the rigidity of the absorbent sheet. In addition, shifting of the materials (such as the absorbent body) can be suppressed inside the absorbent sheet, thereby suppressing the twisting of the absorbent sheet.

### Aspect 9

The absorbent sheet according to any one of Aspects 1 to 8, wherein, in a central portion of the absorbent sheet in the longitudinal direction and the lateral direction, a compressed portion is not provided in which at least the absorbent core is compressed partially in the planar direction and in the thickness direction.

According to the absorbent sheet of Aspect 9, it can be suppressed that, in the central portion of the absorbent sheet where excretion is likely to occur, the sheet is folded due to the compressed portion that is partially compressed. This can ensure the absorption area. Thus, the leakage from the absorbent sheet can be suppressed.

### Aspect 10

The absorbent sheet according to any one of claims 1 to 9, wherein, a density of a portion of the absorbent sheet where the absorbent core is present is 0.35 g/cm³ or less.

According to the absorbent sheet of Aspect 10, as compared with the case where the density of the absorbent sheet is greater than 0.35 g/cm³, a crease for packaging is less likely to be formed, which can suppress the twisting of the absorbent sheet due to the crease. In addition, the absorbent sheet can be placed flat.

### Aspect 11

The absorbent sheet according to any one of Aspects 1 to 10, wherein, the absorbent core extends to a longitudinal side-edge portion of the absorbent sheet, and, the absorbent core extends to a lateral side-edge portion of the absorbent sheet, the longitudinal side-edge portion being a region extending inwardly from a longitudinal side edge of the absorbent sheet by a distance equal to an average thickness of the absorbent sheet, the lateral side-edge portion being a region extending inwardly from a lateral side edge of the absorbent sheet by a distance equal to an average thickness of the absorbent sheet.

According to the absorbent sheet of Aspect 11, the absorbent core having high rigidity extends to the side-edge portions, thereby increasing the rigidity of the absorbent sheet in the large region of the absorbent sheet in the planar direction. This can suppress the twisting of the absorbent sheet. Therefore, the absorption area can be ensured to suppress the leakage from the absorbent sheet.

### Aspect 12

The absorbent sheet according to any one of Aspects 1 to 11, wherein, the absorbent sheet is divided into regions each having a longitudinal length of 40 mm and a lateral length of 40 mm, the regions throughout which the absorbent core is present are defined as second divided regions, and, a CV value that indicates variation in weight of the second divided regions is 10% or less.

According to the absorbent sheet of Aspect 12, the folding of the sheet due to the difference in weight can be suppressed, as compared with the case where the CV value indicating variation in weight of the second divided regions is greater than 10%. This can suppress the twisting of the absorbent sheet. Since the variation in weight of the absorbent core is small, the excreted liquid and the like can be absorbed and retained regardless of the position of the absorbent sheet. This can suppress the leakage from the absorbent sheet.

### Embodiment

Hereinafter, an embodiment of an absorbent sheet according to the present disclosure will be described. The absorbent sheet according to the present disclosure is applicable to, for example, an animal absorbent sheet that disposes of excrement from animals such as pets. In this case, the absorbent sheet can be used alone, or can be placed in an animal litter box to use it. In addition, the absorbent sheet of the present disclosure can be used for various purposes, such as medical sheets, childcare sheets, and nursing sheets used in changing diapers, used for preventing dirt of bedding and the like.

### Basic Configuration of Absorbent Sheet 1

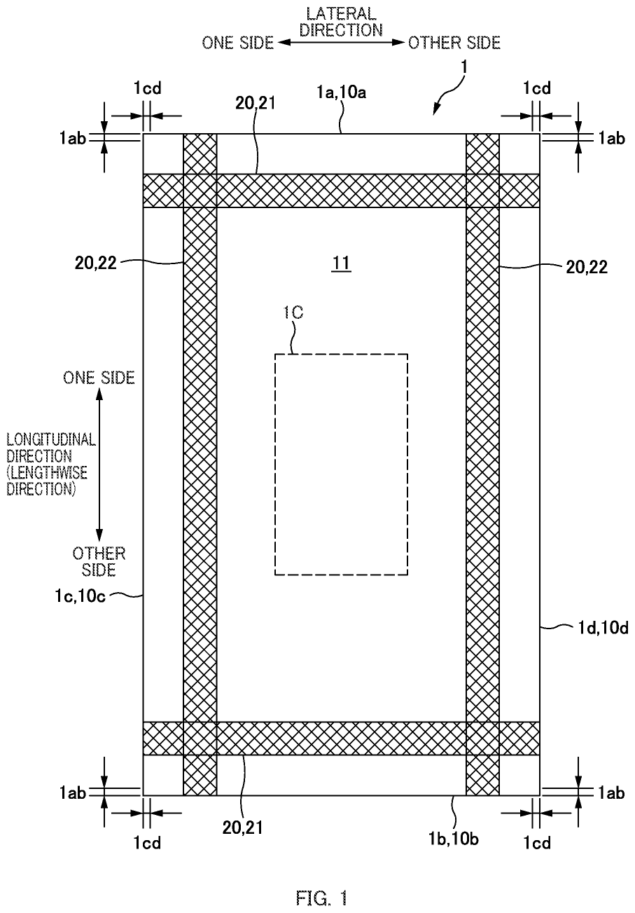

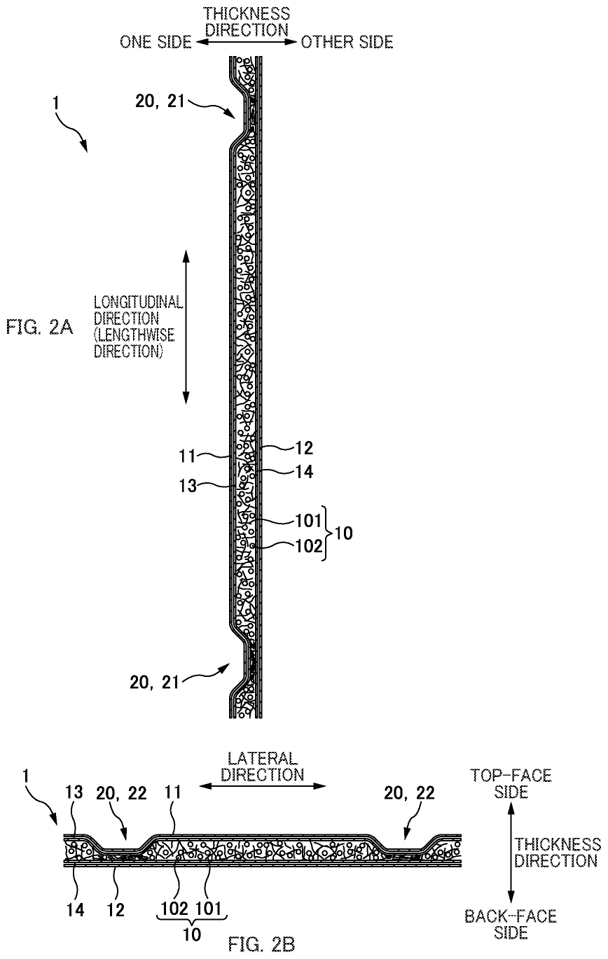

FIG. 1 is a plan view of an absorbent sheet 1 as viewed in the thickness direction. FIG. 2A and FIG. 2B are each a side view of the absorbent sheet 1, taken along the thickness direction. FIG. 2A is a side view of the absorbent sheet 1 at lateral side edges 1c, 1d thereof, and FIG. 2B is a side view of the absorbent sheet at longitudinal side edges 1a, 1b thereof. FIG. 2A is a common view illustrating a side surface at the side edge 1c on a lateral-one side of the absorbent sheet 1 and a side surface at the side edge 1d on a lateral-other side of the absorbent sheet 1. FIG. 2B is a common view illustrating a side surface at the side edge 1a on a longitudinal-one side pf the absorbent sheet 1 and a side surface at the side edge 1b on a longitudinal-other side of the absorbent sheet 1.

The absorbent sheet 1 is a sheet having the longitudinal direction, the lateral direction, and the thickness direction that are orthogonal to each other, and has a rectangular planar shape when viewed in the thickness direction. The planar shape of the absorbent sheet 1 is not limited to a rectangular shape, and may be various shapes such as a square, a substantially quadrilateral (e.g., a quadrilateral with rounded corners or convexly-curved side edges), a polygon, and an ellipse. In the case where the longitudinal length and the lateral length of the absorbent sheet 1 are different from each other as in the present embodiment, the lengthwise direction of the absorbent sheet 1 is defined as a longitudinal direction, and the transverse direction of the absorbent sheet 1 is defined as a lateral direction. In the case where the absorbent sheet 1 has a shape other than the rectangular shape, the linear direction along the longest portion of the absorbent sheet 1 is defined as the longitudinal direction (lengthwise direction), and the direction orthogonal to the longitudinal direction is defined as the lateral direction.

The absorbent sheet 1 includes an absorbent core 10, a liquid-permeable top-face sheet 11 arranged on the top-face side in the thickness direction with respect to the absorbent core 10, and a back-face sheet 12 arranged on the back-face side in the thickness direction with respect to the absorbent core 10. The absorbent core 10 is formed by mixing superabsorbent polymer 102 (SAP) into an aggregate body of liquid-absorbent fibers 101. Examples of the liquid-absorbent fibers 101 include cellulosic fibers (e.g., pulp fibers, non-wood fibers such as cotton or hemp, and regenerated fibers such as rayon), and hydrophilized thermoplastic synthetic fibers (e.g., polyethylene and polypropylene). Examples of the liquid-permeable top-face sheet 11 include a nonwoven fabric, a perforated film, and the like. Examples of the back-face sheet 12 include a liquid-impermeable sheet such as a resin film, and a semi-liquid-impermeable sheet such as an SMS nonwoven fabric sheet and water-repellent nonwoven fabric sheet.

The absorbent sheet 1 may be a sheet capable of absorbing liquid on both surfaces thereof, and the back-face sheet 12 may be a liquid-permeable sheet as with the top-face sheet 11. Such an absorbent sheet 1 may be used as an auxiliary absorbent article that absorbs excreted fluid, in combination with another absorbent article or absorbent sheet.

The absorbent sheet 1 includes a core-wrapping sheet 13 (which is also referred to as a first core-wrapping sheet 13) that covers the top-side surface of the absorbent core 10 in the thickness direction, and a core-wrapping sheet 14 (which is also referred to as a second core-wrapping sheet 14) that covers the back-side surface of the absorbent core 10. A stacked body formed by stacking the core-wrapping sheets 13, 14 on the absorbent core 10 is also referred to as an absorbent body 2. The core-wrapping sheets 13, 14 are liquid-permeable sheets. Examples of the core-wrapping sheets 13, 14 include a tissue, a nonwoven fabric sheet, and a perforated film.

The absorbent sheet 1 has, in a part of the plane thereof, compressed portions 20 in which at least the absorbent core 10 is compressed in the thickness direction. The compressed portions 20 are portions in which the absorbent core 10 is recessed in the thickness direction, and is thus thinner than in the surrounding regions. The density of the absorbent core 10 (liquid-absorbent fibers 101) is also higher than in the surrounding regions.

In the compressed portions 20 illustrated in FIG. 2A and the like, the entire absorbent sheet 1 is compressed from the top-face sheet 11 side. However, the configuration is not limited thereto. For example, the following configuration of the compressed portions 20 is acceptable in which: only the absorbent core 10 is compressed; only the absorbent core 10 and the core-wrapping sheets 13, 14 are compressed; or the absorbent sheet 1 is compressed from the back-face sheet 12 side.

In the compressed portions 20, the region having the compressed portions 20 may be compressed uniformly (with a groove shape), or may be compressed in a predetermined pattern. Examples of the region having the compressed portions 20 compressed in the predetermined pattern include the region having a plurality of small-compressed portions discretely arranged or a plurality of small-compressed portions arranged in a line-like manner. The region having the compressed portions 20 may have a plurality of types of compressed portions with different compression strengths (depths of the recesses).

### Method of Manufacturing Absorbent Sheet 1

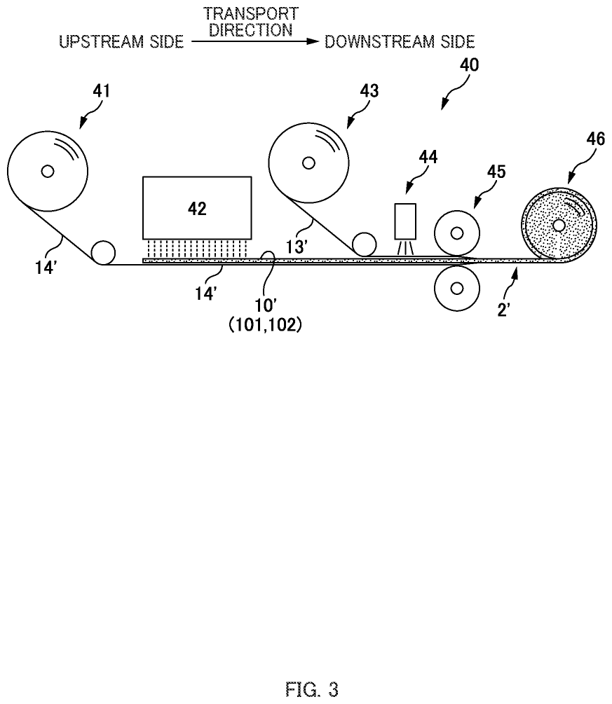

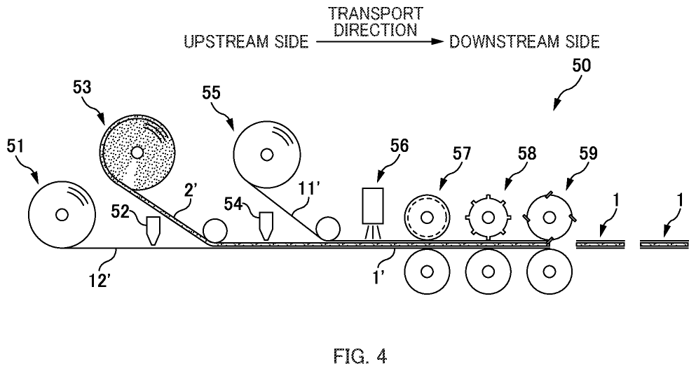

FIG. 3 is an illustrative diagram of a method of manufacturing an absorbent-body continuous body 2′. FIG. 4 is an illustrative diagram of a method of manufacturing the absorbent sheet 1. Next, an example of a method of manufacturing the absorbent sheet 1 will be described. In the manufacturing apparatus 40 and 50 shown in FIGS. 3 and 4, the lateral direction (transverse direction) of the absorbent sheet 1 (absorbent core 10) corresponds to the transport direction (MD direction) of the manufacturing apparatus 40 and 50, and the longitudinal direction (lengthwise direction) of the absorbent sheet 1 corresponds to the CD direction which intersects with the transport direction of the manufacturing apparatus 40 and 50. However, the present disclosure is not limited thereto, and the directions may correspond vice versa.

FIG. 3 illustrates the manufacturing apparatus 40 for manufacturing the absorbent-body continuous body 2′. A first-core-wrapping-sheet continuous body 13′, a absorbent-core continuous body 10′, and a second-core-wrapping-sheet continuous body 14′, which are stacked together, are collectively referred to as the absorbent-body continuous body 2′. The manufacturing apparatus 40 includes supply rollers 41, 43 that supply materials, an air laid machine 42, a water spray 44, press rollers 45, and a wind-up roller 46.

In the method of manufacturing the absorbent-body continuous body 2′, the second-core-wrapping-sheet continuous body 14′ is supplied to a manufacturing line by the supply roller 41. On the second-core-wrapping-sheet continuous body 14′ to be transported in the transport direction by an unillustrated transport device (e.g., a transport belt or drive roller), the absorbent core 10 (the liquid-absorbent fibers 101 and the superabsorbent polymer 102) are deposited by the air laid machine 42. The absorbent core 10 is continuously supplied from the air laid machine 42. In FIG. 3, the second-core-wrapping-sheet continuous body 14′ is transported horizontally, but the present disclosure is not limited thereto, and the second-core-wrapping-sheet continuous body 14′ may be transported in the state of being wrapped around a rotating drum.

Next, the first-core-wrapping-sheet continuous body 13′ is supplied onto the absorbent-core continuous body 10′ by the supply roller 43. Subsequently, water is ejected by the water spray 44 to a stacked body that is constituted by the first-core-wrapping-sheet continuous body 13′, the absorbent-core continuous body 10′, and the second-core-wrapping-sheet continuous body 14′. Thereafter, the press rollers 45 evenly press the entire surface of the stacked body in the thickness direction. The press rollers 45 being a pair of rollers may be heated or non-heated, but the heated rollers can strongly press the stacked body. The location of the water spray 44 is not limited to that shown in FIG. 3. For example, the water spray 44 may be provided in a location before the supply of the first-core-wrapping-sheet continuous body 13′, or may be provided in a plurality of locations. Water may be ejected from below or from both above and below.

As described above, the absorbent-body continuous body 2′ is manufactured and the absorbent-body continuous body 2′ is wound by the wind-up roller 46. A roll body of the absorbent-body continuous body 2′ is set in the manufacturing apparatus 50 of the absorbent sheet 1 shown in FIG. 4.

The manufacturing apparatus 50 for manufacturing the absorbent sheet 1 includes supply rollers 51, 53, 55, adhesive discharge portions 52, 54 that discharge hot-melt adhesives, a water spray 56, compressed rollers 57, 58, and cut rollers 59.

The method of manufacturing the absorbent sheet 1 will be described as follows. First, the adhesive discharge portion 52 applies an adhesive onto the upper surface of a back-face sheet continuous body 12′ that is supplied from the supply roller 51, and the absorbent-body continuous body 2′ that is supplied from the supply roller 53 is stacked on such an upper surface of the back-face sheet continuous body 12′ onto which the adhesive has been applied. Furthermore, the adhesive discharge portion 54 applies an adhesive onto the upper surface of the absorbent-body continuous body 2′, and top-face sheet continuous body 11′ supplied from the supply roller 55 is stacked on such an upper surface of the absorbent-body continuous body 2′ onto which the adhesive has been applied. Thus, a stacked-body continuous body 1′ is formed by stacking the top-face sheet continuous body 11′ that is continuous in the transport direction, the absorbent-core continuous body 2′ that is continuous in the transport direction, and the back-face sheet continuous body 12′ that is continuous in the transport direction.

Next, water is ejected onto the stacked-body continuous body 1′ by the water spray 56. As a result, hydrogen-bonding occurs between cellulosic materials. This can manufacture the absorbent sheet 1 with its constituent materials firmly bonded together. The location and number of the water spray 56 are not limited to those shown in FIG. 4.

Next, laterally-compressed portions 21 are formed by the compression roller 57, and longitudinally-compressed portions 22 are formed by the compression roller 58. The compression rollers 57, 58 are constituted by a pair of rollers, respectively, and protruding portions corresponding to the shape of each compressed portion 20 are formed on the outer circumferential surface of one of or both the pair of rollers. The compression rollers 57, 58 may be heated or non-heated, but the heated rollers can strongly compress the stacked body.

Finally, the cut rollers 59 cut the stacked-body continuous body 1′. The cut rollers 59 are constituted by a pair of rollers; one of the rollers having a cutter blade corresponding to the cutting position on its outer circumferential surface, and the other of the rollers receiving the cutter blade of the one of the rollers. At this time, the cut rollers 59 cut the stacked-body continuous body 1′ such that the length of the stacked-body continuous body 1′ in the transport direction is equal to the longitudinal length or lateral length of the absorbent sheet 1 (here, the lateral length), while transporting the stacked-body continuous body 1′ in the transport direction.

The above-mentioned manufacturing method is merely an example, and the present disclosure is not limited thereto. For example, the manufacturing method may include a folding process in which the stacked-body continuous body 1′ is cut to a product size by the cut roller 59 and then folded to a packaging size, or a folding process for packaging before the stacked-body continuous body 1′ is cut by the cut roller 59. In addition, the rectangular absorbent sheet 1 need not be folded to be distributed. For example, the absorbent sheet 1 that is continuous in a predetermined direction and wound onto a roll may be distributed so as to cut such a rolled absorbent sheet 1 to an appropriate size by a user at a time of use.

### Absorbent Sheet 1 that Suppresses Leakage

In the present embodiment, the maximum thickness of the absorbent sheet 1 is 2.0 mm or less, and the average value of the bending rigidity B of the absorbent sheet 1 in the longitudinal direction according to the KES method is 0.5 gf·cm²/cm or more, and the average value of the bending rigidity B of the absorbent sheet 1 in the lateral direction according to the KES method is 0.5 gf·cm²/cm or more.

Thus, the absorbent sheet 1 of the present embodiment is less likely to be twisted, as compared with the case where the average value of the bending rigidity B of the absorbent sheet in the longitudinal direction and the average value of the bending rigidity B of the absorbent sheet in the lateral direction are less than 0.5 gf·cm²/ cm. In addition, in the absorbent sheet, in the case where the average value of the bending rigidity B in one of the longitudinal direction and the lateral direction is 0.5 gf·cm²/ cm or more but the average value of the bending rigidity B in the other direction of the longitudinal direction and the lateral direction is less than 0.5 gf·cm²/cm, such an absorbent sheet is likely to be twisted when a force in the other direction is applied. As compared with the above-mentioned case, the absorbent sheet 1 of the present embodiment has high rigidity in both the longitudinal direction and the lateral direction. Therefore, the absorbent sheet 1 is less likely to be twisted even when forces in various directions are applied. Thus, even when a user (pet such as a dog and cat, child, and care receiver) moves on the absorbent sheet 1, the absorbent sheet 1 is less likely to be twisted (less likely to be crumpled or wrinkled), which ensures an absorption area in which the excreted fluid can be received and absorbed. For this reason, leakage of the excreted fluid from the absorbent sheet 1 can be suppressed.

In addition, the maximum thickness of the absorbent sheet 1 of the present embodiment is 2.0 mm or less. Thus, a height difference is less likely to be caused between the absorbent sheet 1 and the placement surface of the absorbent sheet 1, as compared with the case where the maximum thickness of the absorbent sheet 1 is greater than 2.0 mm. This can suppress curling of the absorbent sheet 1 due to pet's legs or the like becoming caught on the absorbent sheet 1 to prevent the absorption area from being covered with the curled portion of the absorbent sheet 1. As a result, the absorption area is ensured, which can suppress the leakage of the excreted fluid from the absorbent sheet 1. In addition, since the maximum thickness of the absorbent sheet 1 is reduced, the absorbent sheet 1 is unlikely to become bulky during distribution and storage, thereby improving handling.

Generally, in order to increase the bending rigidity of the absorbent sheet, constituent materials of the absorbent sheet are added or the basis weight of the constituent materials increases. However, in such a way, the thickness of the absorbent sheet increases. In contrast, in the absorbent sheet 1 of the present embodiment, the maximum thickness is reduced to suppress curling of the absorbent sheet 1 while improving the bending rigidity in the longitudinal direction and the lateral direction. This also suppresses the twist (winkles occurrence) of the absorbent sheet to ensure the absorption area. As a result, the leakage of the excreted fluid from the absorbent sheet 1 can be suppressed.

As described above, in the present embodiment, the absorbent sheet 1 is thin, has high rigidity, and is integrated in the thickness direction. Therefore, even when the user cuts the absorbent sheet 1 with scissors or the like, the absorbent core 10 is less likely to spread. Then, the absorbent sheet 1 can be cut to a desired size for use.

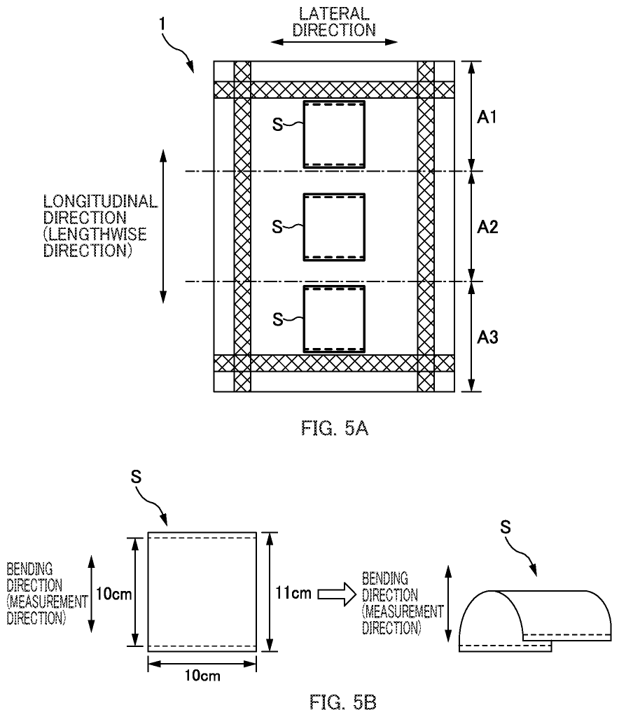

FIG. 5A and FIG. 5B are each an illustrative diagram of a method of measuring the bending rigidity B. The bending rigidity B (gf·cm²/cm) of the absorbent sheet 1 according to the KES method can be measured by a known method. For example, the measurement is performed using Large bending Tester, KES-FB2-D manufactured by Kato Tech Co., Ltd. or equivalent thereof.

First, two absorbent sheets 1 (in a dry state before use) are prepared. The two absorbent sheets 1 have the same configuration and are manufactured using the same manufacturing method. As shown in FIG. 5A, the absorbent sheet 1 is unfolded and stretched without wrinkles, and then divided into three regions at the maximum length in the longitudinal direction (lengthwise direction). Such three divided regions correspond to regions A1 to A3, respectively. One sample S is cut out from each region A1 to A3. Three samples S are cut out from a single absorbent sheet 1 (FIG. 5A) to measure the bending rigidity B in the longitudinal direction, and three samples S are cut out from another single absorbent sheet 1 (unillustrated) to measure the bending rigidity B in the lateral direction. As shown in FIG. 5B, the length of each sample S in the measurement direction of the bending rigidity B is 110 mm (including the length of 5 mm to be held by chucks, at each of upper and lower portions of the sample S), and the length of each sample S in the direction orthogonal to the measurement direction of the bending rigidity B is 100 mm. In addition, each sample S is cut out such that the absorbent core 10 is present in the entire region of each sample S. When each sample S is cut out, the sample S is cut out so as not to include the folded portion for packaging. Unlike the absorbent sheet 1 of the present embodiment, in the case where the compressed portions 20 are provided on a part of the plane, not on the entire surface of the sheet, each sample S is cut out so as not to include the compressed portions 20.

Next, both end portions of each sample S in the measurement direction thereof (bending direction) are held and fixed by the measurement tester's chucks that are horizontally spaced by 110 mm, and the sample S is then bent in the measurement direction as shown in FIG. 5B. At this time, the sample S is bent to the top-face side to a maximum curvature of +0.5 cm⁻¹ at a deformation speed of 0.1 cm⁻¹/sec, is then bent to the back-face side to a maximum curvature of −0.5 cm⁻¹, and is thereafter returned to the original position. The bending rigidity B (gf·cm²/cm) is calculated from the average value of: the slope of the bending moment M with respect to curvature K=0.1 to 0.3 (slope of M-K curvature) when bent toward the top-face side; and the slope of the bending moment M with respect to curvature K=−0.1 to −0.3 (slope of M-K curvature) when bent toward back side.

As described above, the bending rigidity B in the longitudinal direction serving as the measurement direction is obtained for each the three samples S, and the average value of the bending rigidity B of the three samples S is referred to as an “average value of the bending rigidity B of the absorbent sheet 1 in the longitudinal direction according to the KES method”. Similarly, the bending rigidity B in the lateral direction serving as the measurement direction is obtained for each of the three samples S, and the average value of the bending rigidity B of the three samples S is referred to as an “average value of the bending rigidity B of the absorbent sheet 1 in the lateral direction according to the KES method”.

The maximum thickness of the absorbent sheet 1 can also be measured using a known method. For example, the measurement is performed using “J-B”, a large dial thickness gauge with spring, manufactured by OZAKI MFG. CO., LTD., or equivalent thereof. The thickness of the absorbent sheet 1 is measured using six samples S that are cut out for measurement of the bending rigidity B. The thickness measurement location is set at a location where the absorbent core 10 is present, the location being spaced away from the folding portion for packaging. In addition, the measurement is performed so as to measure the thickness (maximum thickness) of a non-compressed portion that is not the compressed portion partially compressed in the planar direction, while avoiding the compressed portion that is larger than measurement terminal. Then, the measurement is performed by applying the pressure of 7.4 gf/cm² to the set measurement location using a measurement terminal with a diameter of 50 mm. In this way, the thicknesses of the six samples S are obtained. The maximum value of these thicknesses is referred to as a “maximum thickness of the absorbent sheet 1”.

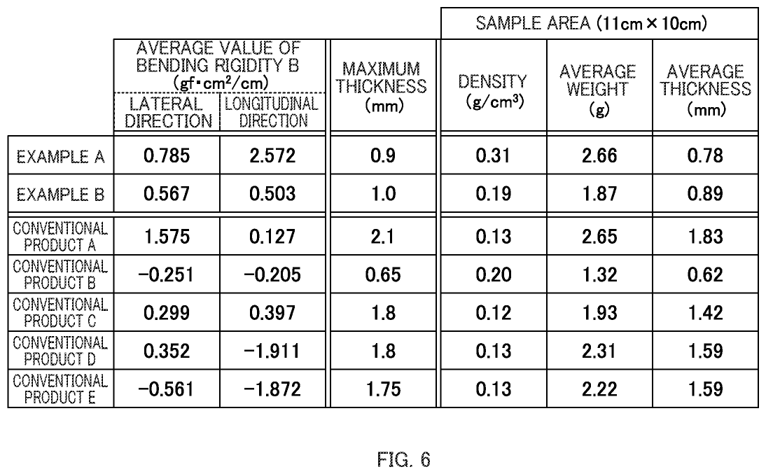

FIG. 6 is a diagram indicating the measurement results of the absorbent sheet 1 of the present embodiment (Examples A, B) and Conventional Products A to E. The absorbent sheet 1 of each Example A, B is manufactured by using the manufacturing method shown in FIGS. 3, 4. Conventional Products A to Care each the existing absorbent sheet that is commercially available as a pet absorbent sheet. Conventional Products D, E are each the existing absorbent sheet manufactured by the applicant.

Specifically, in Example A, the basis weight of the liquid-absorbent fiber (pulp fiber) constituting the absorbent core 10 was set to the same as the basis weight of the pulp fiber in Conventional Product D. The basis weight of the superabsorbent polymer 102 blended with the pulp fiber was the same as the basis weight of the superabsorbent polymer in Conventional Product D. In addition, the core-wrapping sheets 13, 14 were tissues, the top-face sheet 11 was an air-through nonwoven fabric sheet, and the back-face sheet 12 was a resin film. Example B and Example A are different in terms of the basis weight. In Example B, the basis weight of the pulp fiber and the basis weight of the superabsorbent polymer 102 were lower than those in Example A. In order to obtain the same thickness in both Example A and Example B, the pressure by the flat pressing during the manufacture of the absorbent core (pressure in the thickness direction by the press rollers 45 shown in FIG. 3) was appropriately adjusted.

Based on the above-mentioned measurement method, the average value of the bending rigidity B and the maximum thickness were measured for Examples A, B and Conventional Products A to E. In Examples A, B, the maximum thickness was 2.0 mm or less, but the average value of the bending rigidity B in the longitudinal direction and the average value of the bending rigidity B in the lateral direction were 0.5 gf·cm²/cm or more. On the other hand, in Conventional Products B to E, the average value of the bending rigidity B in the longitudinal direction and the average value of the bending rigidity B in the lateral direction were less than 0.5 gf·cm²/cm. In Conventional Product A, the average value of the bending rigidity B in the lateral direction was 0.5 gf·cm²/cm or more, but the average value of the bending rigidity B in the longitudinal direction was less than 0.5 gf·cm²/cm.

The above results show that, in both Examples A, B, the absorbent sheet 1 has higher rigidity in both the longitudinal direction and the lateral direction, and is thus less likely to be twisted (less likely to be crumpled or wrinkled) when forces in various directions are applied, as compared with Conventional Products A to E. Therefore, it can be seen that the absorption area is ensured to suppress the leakage. In addition, the maximum thickness of the absorbent sheet 1 in each Example A, B was small and equivalent to those of Conventional Products A to E, and was smaller than the maximum thickness (thickness of 1.5 mm or more) of most of Conventional Products except for Conventional Product B. Thus, it can be seen that, in the absorbent sheet 1 of each Example A, B, the curling of the absorbent sheet 1 due to the pet becoming caught on the absorbent sheet 1 can be suppressed to ensure the absorption area and suppress the leakage.

In addition, the absorbent core in each Conventional Product D, E manufactured by the applicant was manufactured using a pattern drum that has been known as the general manufacturing apparatus (unillustrated) of the absorbent core. The pattern drum is a rotating drum having plurality of molds (recessed portions) on its outer circumferential surface at predetermined intervals, the molds each having a shape which corresponds to shape of the absorbent core. Suction holes are provided on the bottom surface of each mold, and liquid-absorbent fiber (pulp fiber) and superabsorbent polymer scattered from a duct facing the pattern drum are deposited on the mold. The absorbent core deposited on the mold is sequentially released from the pattern drum and transferred onto a core-wrapping sheet or the like.

In the case of the method of manufacturing the absorbent core using the pattern drum, there are cases where the absorbent core cannot be released neatly from the mold, or the liquid-absorbent fiber float or the superabsorbent polymer rolls when the absorbent core is transferred to the core-wrapping sheet. Accordingly, it is difficult to arrange materials uniformly in the planar direction of the absorbent core. As a result, when the entire surface of the absorbent core is pressed, for example, a force cannot be applied uniformly in the planar direction. This causes variation in thickness to increase the maximum thickness. Furthermore, the absorbent core cannot be compressed completely in the thickness direction, and its density cannot be increased. As a result, the rigidity of the absorbent sheet becomes smaller.

In contrast, according to the method for manufacturing the absorbent core 10 illustrated in FIG. 3, the liquid-absorbent fibers 101 and the superabsorbent polymer 102 are deposited on the core-wrapping sheet. Thus, the problem caused by using the conventional pattern drum (e.g., the problem in which the absorbent core cannot be neatly die-cut, or in which the variation in materials occurs at a time of the transfer of the absorbent core) are less likely to occur. Therefore, the superabsorbent polymer 102 is blended with the liquid-absorbent fibers 101 uniformly in a dispersed manner, and the blended superabsorbent polymer 102 and the liquid-absorbent fibers 101 are then uniformly distributed on the core-wrapping sheet.

Therefore, when the press rollers 45 shown in FIG. 3 apply the pressure to the absorbent core 10 in the thickness direction, the force is likely to be applied evenly to the absorbent core 10. As a result, the thickness is less likely to vary to reduce the maximum thickness. In addition, the density of the absorbent core 10 can be increased, which can firmly join the materials together. This increases the rigidity of the absorbent sheet 1. In addition, regardless of the MD direction (transport direction) and CD direction during the manufacture, the imbalance of materials in the planar direction of the absorbent core 10 is suppressed, thereby improving the rigidity in both the longitudinal direction and the lateral direction of the absorbent sheet 1. However, the manufacturing method shown in FIGS. 3 and 4 is merely an example, and the manufacturing method of the absorbent sheet 1 of the present embodiment is not limited thereto. The absorbent core 10 of the present embodiment may be manufactured using a pattern drum, for example.

In the present embodiment, it is preferable that the density of the absorbent sheet 1 in a portion where the absorbent core 10 is present is 0.35 g/cm³ or less. Accordingly, as compared with the case where the density of the absorbent sheet is higher than 0.35 g/cm³, a crease is less likely to be formed at the folded portion for packaging, which can suppress that such a crease causes the absorbent sheet 1 to be folded or twisted (winkled). This can ensure the absorption area of the absorbent sheet 1. Since the crease is less likely to be formed, the absorbent sheet 1 can be also placed flat. As a result, a gap is less likely to be formed between the absorbent sheet 1 and the placement surface, which can suppress curing of the absorbent sheet 1 due to the pet's legs becoming caught on such a gap. This ensures the absorption area of the absorbent sheet 1. When the absorbent sheet 1 is placed in an animal litter box, the absorbent sheet 1 can be also easily folded to fit the shape of the litter box (tray), thereby easily perform the placement of the absorbent sheet 1.

In addition, in the present embodiment, the rigidity of the absorbent sheet 1 is increased while reducing the maximum thickness of the absorbent sheet 1. Then, the density of the absorbent sheet 1 is reduced to 0.35 g/cm³ or less, without increasing the basis weight (g/cm²) of the absorbent core 10 to increase the density. Therefore, the basis weight of the absorbent core 10 is not increased more than necessary, exceeding the absorption amount (water retention) required for the absorbent sheet 1. This leads to material reduction and cost reduction. However, the configuration is not limited thereto. The density of the absorbent sheet 1 may be greater than 0.35 /cm³.

The density (g/cm³) can be measured using a known method. For example, the thicknesses (mm) of the six samples S that are cut out at a time of measurement of the maximum thickness of the absorbent sheet 1 are used to calculate the average value of such thicknesses. In addition, the weight (g) of each of the six samples S is measured and the average value thereof is calculated. The density can be calculated based on the average weight (g), the average thickness (mm) and each area (110 mm×1.00 mm) of the samples S.

FIG. 6 shows the results of actual density measurement in Examples A, B and Conventional Products A to E. Similarly to Conventional Products A to E, in Examples A, B, the density is reduced to 0.35 g/cm³ or less. Thus, in the absorbent sheet 1 of the present embodiment (Examples A, B) the rigidity is increased while reducing the maximum thickness, but the density is not excessively higher as compared with Conventional Products A to E. Therefore, it can be seen that the basis weight of the absorbent core 10 is not unnecessarily high.

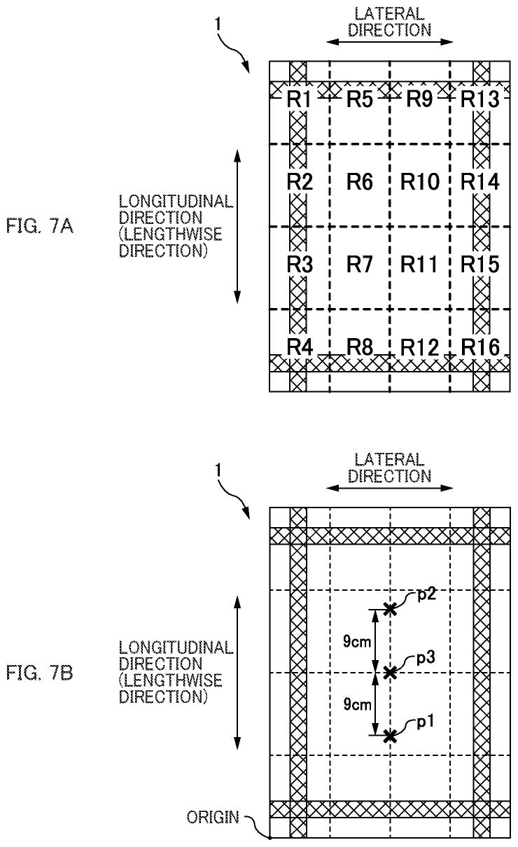

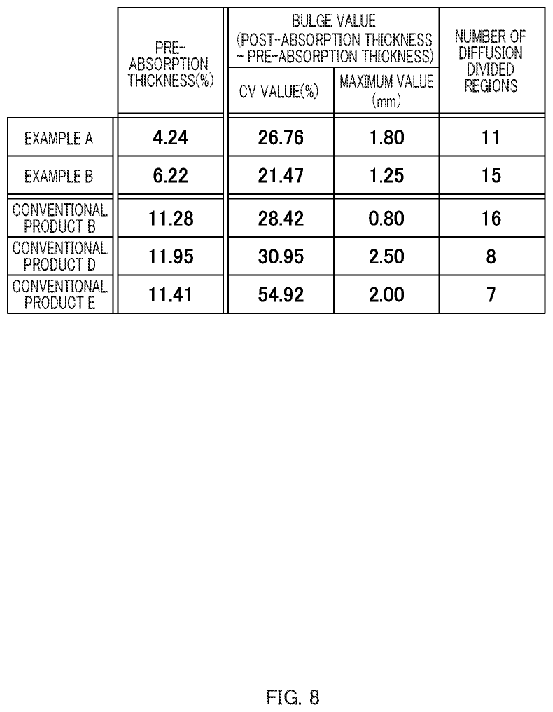

FIG. 7A is an illustrative diagram of divided regions R1 to R16, and FIG. 7B is an illustrative diagram of a drop test of artificial urine. FIG. 8 is a diagram illustrating the variation in thickness and the results of the drop test. The following thickness measurements were performed for the above-mentioned Examples A, B and Conventional Products B, D, E. As shown in FIG. 7A, the absorbent sheet is divided into four portions in the longitudinal direction and divided into four portions in the lateral direction, and such divided portions are defined as “divided regions R1 to R16”.

In the absorbent sheet 1 before absorption, at a time of measurement of the thickness of the absorbent sheet 1 in each divided region R1 to R16 where the absorbent core 10 is present, it is preferable that a CV value indicating variation in thickness in the sixteen divided regions R1 to R16 is 10% or less.

Accordingly, variation in thickness of the absorbent sheet 1 before absorption is smaller and the surface unevenness is smaller than the case where the CV value of the thickness in the divided regions R1 to R16 is higher than 10%. This can suppress that the absorbent sheet 1 is folded due to the thickness difference, and that the pet's legs become caught on protruded portions. Thus, the twisting (winkles occurrence) of the absorbent sheet 1 can be suppressed. As a result, the absorption area of the absorbent sheet 1 can be ensured, and the leakage of the excreted fluid from the absorbent sheet 1 can be suppressed.

The measurement method of the thicknesses in the divided regions R1 to R16 is the same as the above-mentioned measurement method of the maximum thickness of the absorbent sheet 1. Then, the central portion (a portion where the absorbent core 10 is present) in each of the divided regions R1 to R16 is measured. The measurement is performed so as to measure the thickness of a non-compressed portion that is not the compressed portion partially compressed in the planar direction, while avoiding the compressed portion that is larger than a measurement terminal. Then, the standard deviation and average value of the thickness of each of the sixteen divided regions R1 to R16 are calculated to obtain the CV value by dividing the standard deviation by the average value (standard deviation/average value).

FIG. 8 shows the actual measurement results of the CV values of the thicknesses in Examples A, B and Conventional Products D, E. The CV value in Example A was 4.24%, the CV value in Example B was 6.22%, the CV value in Conventional Product B was 11.28%, the CV value in Conventional Product D was 11.95%, and the CV value in Conventional Product was 11.41%. Based on the above results, in the absorbent sheet 1 of the present embodiment (Examples A, B), the CV value of the thickness before absorption was 10% or less. Then, the results showed that variation in thickness was smaller to make the absorbent sheet less likely to be twisted, as compared with Conventional Products B, D, and E. As mentioned above, this reason can be considered as follows: in the absorbent sheet 1 of the present embodiment, the absorbent core 10 is disposed uniformly in the planar direction, which makes the pressure likely to be applied uniformly to the absorbent sheet 1 at a time of pressing during the manufacture to reduce variation in thickness. However, the configuration is not limited thereto. The CV value of the thickness of the absorbent sheet 1 before absorption may be greater than 10%.

When the absorbent sheet 1 is subjected to the “drop test” performed by dropping the artificial urine at a predetermined position on the absorbent sheet 1, the divided regions R1 to R16 where the artificial urine has been diffused in the absorbent sheet 1 subjected to the drop test are referred to as “diffusion divided regions”. Then, the absorbent sheet 1 before the drop test has a “pre-absorption thickness” that is the thickness of portion of each diffusion divided region where the absorbent core 10 is present. The absorbent sheet 1 after the drop test has a “post-absorption thickness” that is the thickness of a portion of each diffusion divided region where the absorbent core 10 is present. It is preferable that the CV value indicating variation in the value obtained by subtracting the pre-absorption thickness from the post-absorption thickness in each diffusion divided region (e.g., the value obtained by subtracting the pre-absorption thickness in the divided region R1 from the post-absorption thickness in the divided region R1), is 30% or less. In the following description, the value obtained by subtracting the pre-absorption thickness from the post-absorption thickness is also referred to as a “bulge value”. The CV value is calculated without including the bulge value (=0) of the divided region where the artificial urine has not diffused.

This reduces the variation in the thickness difference before and after absorption, as compared with the case where the CV value of the bulge value (=post-absorption thickness-pre-absorption thickness) is greater than 30%. As a result, the unevenness on the sheet surface is likely to be reduced. Thus, even after absorption of the excreted fluid, it can be suppressed that the pet's legs become caught on the protruded portions due to the thickness difference, which can suppress twisting of the absorbent sheet 1. Therefore, even when the repeated excretion occurs onto the absorbent sheet 1, the absorption area is ensured to suppress the leakage of the excreted fluid from the absorbent sheet 1.

It is also preferable that the maximum value of values (bulge values) obtained by subtracting the pre-absorption thickness from the post-absorption thickness in the respective diffusion divided regions is 1 mm more. In the absorbent sheet having the bulge value after absorption being less than 1 mm, the absorption amount (water retention) is small. When the repeated excretion occurs or a large amount of excreted fluid is excreted, the excreted fluid may leak from the absorbent sheet. For this reason, the bulge value after absorption is set to 1 mm or more, which can reliably absorb and retain the excreted fluid on the absorbent sheet 1, as compared with the case where the bulge value is less than 1 mm. As described above, the absorption amount is ensured while keeping the CV value (variation) of the bulge value at 30% or less, which can suppress the leakage of the excreted fluid and also suppress the twisting of the absorbent sheet 1 even after absorption of the excreted fluid.

FIG. 8 shows that the calculation results of the CV value and the maximum value of the bulge value, in the actually-performed drop test. In Example A, the number of diffusion divided regions was eleven, the CV value of the bulge value was 26.76%, and the maximum value of the bulge value was 1.80 mm. In Example B, the number of diffusion divided regions was fifteen, the CV value of the bulge value was 21.47%, and the maximum value of the bulge value was 1.25 mm. In Conventional Product B, the number of diffusion divided regions was sixteen, the CV value of the bulge value was 28.42%, and the maximum value of the bulge value was 0.80 mm. In Conventional Product D, the number of diffusion divided regions was 8, the CV value of the bulge value was 30.95%, and the maximum value of the bulge value was 2.50 mm. In Conventional Product E, the number of diffusion divided regions was seven, the CV value of the bulge value was 54.92%, and the maximum value of the bulge value was 2.00 mm. The above results showed that, in the absorbent sheet 1 of the present embodiment (Examples A, B), the CV value of the bulge value was 30% or less, and the variation in thickness difference before and after absorption was smaller, as compared with Conventional Products D, E, to reduce the unevenness on the sheet surface after absorption, which makes the absorbent sheet 1 less likely to be twisted.

In Conventional Product B, the CV value of the bulge value was 30% or less, however, the maximum value of the bulge value was small, less than 1 mm. Therefore, the small absorption amount may cause the leakage when the repeated excretion occurs, and the like. In contrast, in Examples A, B, the maximum value of the bulge value was 1.00 mm or more. It was found that the absorbent sheet 1 bulges by reliably absorbing and retaining the excreted fluid, which can suppress the leakage. When the surface of Conventional Product B after the actually-performed drop test was pressed with fingers, it was found that liquid return was felt on the surface of Conventional Product B, as compared with Examples A, B.

Furthermore, it is preferable that the maximum value of values (bulge values) obtained by subtracting the pre-absorption thickness from the post-absorption thickness in the diffusion divided regions is 2 mm or more. As a result, the localized bulging does not occur in the absorbent sheet, as compared with the case where the bulge value is greater than 2 mm. This can reduce the thickness difference between the diffusion divided regions and other regions (specifically, portions where the excreted fluid has not diffused). Thus, it is suppressed that the absorbent sheet 1 is folded due to the thickness difference and that the pet's legs become caught on the localized-bulged portion, which can suppress the twisting of the absorbent sheet 1. Then, even in the absorbent sheet 1 after absorption, the absorption area is ensured to suppress the leakage of the excreted fluid.

As shown in the results shown in FIG. 8, in the absorbent sheet 1 of the present embodiment (Examples A, B), the maximum value of the bulge value was 2 mm less. Therefore, it was found that, in the absorbent sheet 1 of the present embodiment, after absorption, localized bulging is less likely to occur, thereby making the twisting of the absorbent sheet 1 less likely to occur. However, the configuration is not limited thereto. The CV value of the bulge value may be smaller than 30%, and the maximum value of the bulge value may be less than 1 mm or greater than 2 mm.

In addition, in Examples A, B and Conventional Products B, D, E, the test was performed for four dogs (1 to 7-year-old, 5 to 10 kg) each using a single absorbent sheet in a dry state. The dogs were observed via video for 24 hours (the dogs excreted (urinated) during the observation). For example, the observation result of the test for a 7-year-old dog weighing 10 kg showed that the sheets of Conventional Products B, D, E were twisted within one hour from the start of the test and remained in a crumpled state. Then, five hours later, the dog urinated onto the crumpled sheet, and the leakage from the sheet was thus caused. On the other hand, in Examples A, B, even after two urinations, the sheet was not twisted, and the leakage from the sheet did not occur. As for other dogs, in Conventional Products B, D, E, the sheets were twisted within 30 minutes from the start of the test, and an immediate replacement repositioning of the sheet was thus required. On the other hand, in Examples A, B, the replacement of the sheet was not required, and the original condition was kept without twisting of the sheet for a longer period of time, as compared with Conventional Products B, D, E. Based on the above-mentioned observation results, it was found that, in the absorbent sheet 1 of the present embodiment (Examples A, B) that has high rigidity and reduced variation in thickness, the absorbent sheet before excretion is less likely to be twisted as compared with Conventional Products B, D, and E. It was also found that, in the absorbent sheet 1 of the present embodiment (Examples A, B) that has: the reduced variation in the bulge value after absorption; and the maximum value of the bulge value being 2 mm or less, the absorbent sheet after excretion is also less likely to be twisted to suppress the leakage from the sheet, as compared with Conventional Products B, D, E.

Here, the method of the drop test will be described. First, in the absorbent sheet to be measured, three drop positions p1 to p3 are determined. As shown in FIG. 7B, a third drop position p3 refers to the central portion of the absorbent sheet (rectangular shape) in the longitudinal direction (lengthwise direction) and lateral direction. A first drop position p1 refers to a position that is 90 mm away from the third drop position p3 on one longitudinally-outward side. A second drop position p2 refers to a position that is 90 mm away from the third drop position p3 on the other longitudinally-outward side. The three drop positions p1 to p3 are located at the same position in the lateral direction (transverse direction).

Next, the absorbent sheet is divided into four regions in the longitudinal direction and into four regions in the lateral direction, and the divided regions R1 to R16 are defined in the absorbent sheet (it is preferable to draw lines with a pen or the like to divide the sheet into regions). The thickness of each of the divided regions R1 to R16 (central portions) to obtain the pre-absorption thickness (the method of measuring the thickness is performed in the same manner as described above.).

Then, 40 cc of artificial urine was dropped onto the first drop position p. After passage of one minute from dropping 40cc of artificial urine onto the first drop position p1, 40 cc of artificial urine was dropped onto the second drop position p2. After passage of one minute from dropping 40 cc of artificial urine onto the second drop position p2, 40 cc of artificial urine was dropped onto the third drop position p3. After passage of one minute from the end of dropping, out of the sixteen divided regions R1 to R16, the regions where the artificial urine has diffused was recognized to determine the diffusion divided regions. Then, the thickness of each diffusion divided region (central portion) was measured to obtain the post-absorption thickness.

Finally, the value obtained by subtracting the pre-absorption thickness in each diffusion divided region from the post-absorption thickness in each diffusion divided region, was calculated as the bulge value. Then, the CV value indicating the variation in the bulge value and the maximum value were obtained.

The artificial urine was prepared by dissolving 200 g of urea, 80 g of sodium chloride, 8 g of magnesium sulfate, 3 g of calcium chloride, and approximately 1 g of dye: Blue 1 in 10 L of ion exchange water.

In the absorbent sheet 1, the core-wrapping sheets 13, 14 are arranged between the absorbent core 10 and at least one of the top-face sheet 11 and the back-face sheet 12 (in the present embodiment, both the top-face sheet 11 and the back-face sheet 12). In this case, it is preferable that the liquid-absorbent fibers 101 constituting the absorbent core 10 include pulp fibers, and the core-wrapping sheets 13, 14 include cellulosic fibers (e.g., pulp fibers, non-wood fibers such as cotton and hemp, regenerated fibers such as rayon). It is preferable that the pulp fibers included in the liquid-absorbent fibers 101 of the absorbent core 10 are hydrogen-bonded with the cellulosic fibers included in the core-wrapping sheets 13, 14.

Accordingly, the absorbent core 10 and the core-wrapping sheets 13, 14 are firmly joined and integrated. This can increase the rigidity of the absorbent sheet 1 to suppress the twisting of the absorbent sheet 1. The shifting between the absorbent core 10 and the core-wrapping sheets 13, 14 in the absorbent sheet 1 can be suppressed, thereby suppressing the twisting (wrinkles occurrence) of the absorbent sheet 1. As a result, the absorption area Of the absorbent sheet 1 is secured, which can suppress the leakage of the excreted fluid from the absorbent sheet 1.

In the manufacturing apparatus 40 illustrated in FIG. 3, the water spray 44 sprays water onto the stacked body formed by stacking the continuous bodies of: the absorbent core 10; and the core-wrapping sheets 13, 14, and then, such a stacked body is flat-pressed by the press rollers 45. As a result, the absorbent core 10 (pulp fiber) is hydrogen-bonded with the core-wrapping sheets 13, 14, and the absorbent sheet 1, which is likely to maintain its hydrogen-bonded state can be manufactured. The pulp fibers in the absorbent core 10 are also hydrogen-bonded with each other, which makes an entangled state of the pulp fibers likely to be maintained.

In the present embodiment, also in the manufacturing apparatus 50 illustrated in FIG. 4, the water spray 56 sprays water onto the stacked body formed by stacking the continuous bodies of the absorbent core 10, the top-face sheet 11, and the back-face sheet 12, and then, the compressed portions 20 are formed. As a result, in the compressed portions 20, the absorbent core 10 (pulp fibers) is more firmly hydrogen-bonded with the core-wrapping sheets 13, 14, and such a hydrogen-bonded state is maintained.

Using a known method can confirm that the liquid-absorbent fibers 101 include the pulp fibers and the core-wrapping sheets 13, 14 include the cellulosic fibers. Examples of the known method include a use of a dye for identification to color the fibers, and observation of the fibers using an optical microscope, and the like. However, the configuration is not limited thereto. The liquid-absorbent fibers 101 need not include the pulp fibers, the core-wrapping sheets 13, 14 need not include the cellulosic fibers, and the absorbent core 10 need not be hydrogen-bonded with the core-wrapping sheets 13, 14.

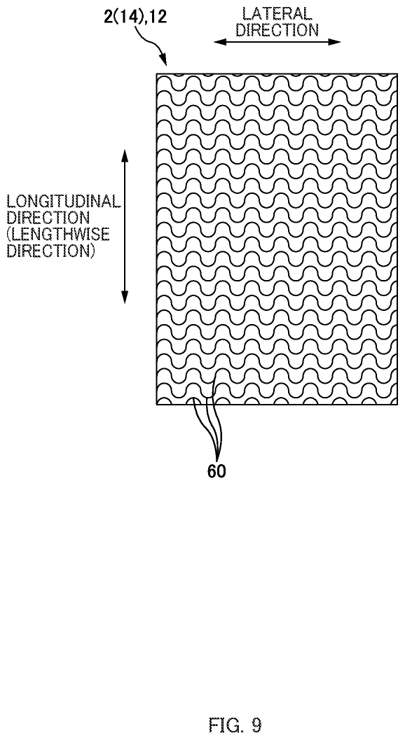

FIG. 9 is a diagram illustrating adhesives 60 that join the back-face sheet 12 and the core-wrapping sheet 14 together. The back surface of the absorbent body 2 including the absorbent core 10, that is, the back surface of the core-wrapping sheet 14 disposed on the back side of the absorbent core 10, is joined onto the back-face sheet 12 by the adhesives 60. The absorbent core 10 need not be covered with the core-wrapping sheets 13, 14. In this case, the absorbent core 10 alone corresponds to the absorbent body, and it is preferable that the back-face sheet 12 is directly joined to the back surface of the absorbent core 10.

The back-face sheet 12 and the back surface the absorbent body 2 are joined together by the adhesives 60. It is preferable that the adhesives 60 are provided continuously in the longitudinal direction over the entire longitudinal range of the back surface of the absorbent body 2 (here, the core-wrapping sheet 14), and are provided continuously in the lateral direction over the entire lateral range of the back surface of the absorbent body 2 (here, the core-wrapping sheet 14).

Accordingly, the back-face sheet 12 and the absorbent body 2 are firmly joined and integrated. This can increase the rigidity of the absorbent sheet 1 to suppress the twisting of the absorbent sheet 1. The application amount of the adhesives 60 is increased as compared with the case where the adhesives 60 are provided discontinuously, thereby also increasing the rigidity of the absorbent sheet 1. The shifting of the back-face sheet 12 and the absorbent body 2 can be suppressed, which can suppress the twisting (winkles occurrence) of the absorbent sheet 1. As a result, the absorption area of the absorbent sheet 1 is secured, which can suppress leakage of the excreted fluid.

Even when the adhesives 60 that join the back-face sheet 12 and the back surface of the absorbent body 2 are applied continuously as described above, the problem of the adhesives 60 inhibiting the absorption of the absorbent core 10 is not caused. Thus, the absorption performance of the absorbent core 10 is ensured. Adhesives (unillustrated) that join the top-face sheet 11 and the top surface (core-wrapping sheet 13) of the absorbent body 2 are provided in the same manner as the adhesives 60 on the back side, thereby further increasing the rigidity of the absorbent sheet 1. Alternatively, the adhesives that join the top-face sheet 11 and the top surface of the absorbent body 2 may be provided discontinuously in the longitudinal direction and the lateral direction, and the application amount (basis weight) of the adhesives may be reduced. This can suppress that the adhesives inhibit the absorption of the absorbent core 10, thereby ensuring the absorption performance of the absorbent core 10.

The adhesives 60 may be applied onto the entire adhesion region in a so-called solid-applied manner, or may be applied in a known application pattern (such as a spiral pattern, an Ω-shaped pattern, and a line pattern). The adhesives 60 being continuous in the predetermined direction (longitudinal direction or lateral direction) means that the adhesives 60 are present entirely in the predetermined direction. For example, FIG. 9 illustrates that the adhesives 60 are applied in the Ω-shaped pattern. In FIG. 9, the adhesives 60 in the Ω-shaped pattern extend continuously in the lateral direction (in the transport direction during the manufacture) and are aligned in a plurality of rows in the longitudinal direction. In such rows, when the longitudinal ends of the adhesives 60 adjacently arranged in the longitudinal direction coincide in position or overlap with each other, it can be said that the adhesives 60 are continuous in the longitudinal direction.

In addition, in the manufacturing apparatus 50 illustrated in FIG. 4, the adhesives 60 are applied onto the back-face sheet 12 (continuous body) by the adhesive discharge portion 52, but the adhesives 60 may be applied onto the core-wrapping sheet 14, or the adhesives 60 may be applied onto both the back-face sheet 12 and the core-wrapping sheet 14.

As shown in FIG. 1, it is preferable that, in the absorbent sheet 1, the compressed portions in which at least the absorbent core 10 is compressed partially in the planar direction and in the thickness direction are not provided in a central portion 1C of the absorbent sheet 1 in the longitudinal direction and the lateral direction. Such compressed portions do not include the portion where the entire surface of the absorbent core 10 is pressed uniformly in the thickness direction (so-called flat-pressed portion). In addition, the central portion 1C in the longitudinal direction and the lateral direction of the absorbent sheet 1 corresponds to the central portion of the absorbent sheet 1 that is divided into three regions at the maximum length in the longitudinal direction, and the central portion of the absorbent sheet 1 that is divided into three regions at the maximum length in the lateral direction.

The rigidity of the absorbent core 10 and the absorbent sheet 1 is increased by the compressed portions (pattern embossing) in which the absorbent core 10 is partially compressed in the planar direction. However, the compressed portions may serve as the folding point of the absorbent sheet 1. The central portion 1C of the absorbent sheet 1 is a portion where the force is more likely to be applied due to pet's position and pet's movement, as compared with the outer peripheral edge portions of the absorbent sheet 1. Thus, the compressed portions are not provided in the central portion 1C of the absorbent sheet 1, which can suppress that the folding of the absorbent sheet 1 along the compressed portions causes the twisting (wrinkles occurrence) of the absorbent sheet 1. In addition, the absorption area of the central portion 1C of the absorbent sheet 1 where excretion is likely to occur can be ensured, which can suppress the leakage of the excreted fluid.

In the present embodiment, the absorbent sheet 1 has the compressed portions 20 (laterally-compressed portions 21 and longitudinally-compressed portions 22) on the outer peripheral edge portions of the absorbent sheet 1, but these compressed portions 20 are less likely to affect the twisting of the entire absorbent sheet 1, as compared with the compressed portions provided in the central portion 1C. Thus, it is preferable that the compressed portions 20 are provided on the outer peripheral edge portions of the absorbent sheet 1 (i.e., outside the central portion 1C, preferably, the outer end portions of the absorbent sheet 1 being divided into four regions in the longitudinal direction and the lateral direction). In this case, the compressed portions 20 (laterally-compressed portions 21 and longitudinally-compressed portions 22) can diffuse the excreted fluid in the lateral direction and the longitudinal direction, which can suppress the leakage of the excreted fluid from the absorbent sheet 1. In addition, the absorbent sheet 1 can be effectively utilized over a wide region in the planar direction. However, the configuration is not limited to thereto. The absorbent sheet 1 need not have the compressed portions 20, or compressed portions may be provided in the central portion 1C of the absorbent sheet 1.

As shown in FIG. 1, longitudinal side-edge portions lab are regions respectively extending inwardly from longitudinal side edges 1a, 1b of the absorbent sheet 1 by the distance equal to the average thickness of the absorbent sheet 1, and lateral side-edge portions 1cd are regions respectively extending inwardly from lateral side edges 1c, 1d of the absorbent sheet 1 by the distance equal to the average thickness of the absorbent sheet 1. In this case, it is preferable that the absorbent core 10 extends to each longitudinal side-edge portion lab of the absorbent sheet 1, and to each lateral side-edge portion 1cd of the absorbent sheet 1. In the absorbent sheet 1 of the present embodiment, the longitudinal side edges 1a, 1b of the absorbent sheet 1 match longitudinal side edges 10a, 10b of the absorbent core 10, and the lateral side edges 1c, 1d of the absorbent sheet 1 match lateral side edges 10c, 10d of the absorbent core 10.

As a result, the absorbent core 10 having high rigidity is present over the large region of the absorbent sheet 1 in the planar direction, thereby increasing the rigidity of the absorbent sheet 1. This can suppress the twisting of the absorbent sheet 1 to ensure the absorption area of the absorbent sheet 1. The length of the sheet extending beyond the absorbent core 10 (such as the top-face sheet 11, the back-face sheet 12, and the core-wrapping sheets 13, 14) is relatively short, which can suppress that the absorption surface is covered by the sheet due to curing of the sheet. This ensures the absorption area. In particular, the length of the back-face sheet 12 extending beyond the absorbent core 10 is shorter than the average thickness of the absorbent sheet 1. As a result, the back-face sheet 12 does not curl and is not folded onto the top-face sheet 11, which ensures the absorption area. Even when the excretion occurs on the outer peripheral edge portions of the absorbent sheet 1, the absorbent core 10 can receive the excreted fluid, thereby making the excreted fluid less likely to leak out of the absorbent sheet 1.

The average thickness of the absorbent sheet 1 can be measured using the same measurement method of the average thickness that has been obtained by measuring the density (g/cm³) described above (see FIG. 6, the average thickness of the six samples S that are cut out for measurement of the bending rigidity B).

In the absorbent sheet 1 of the present embodiment, the longitudinal and lateral side edges 10a to 10d of the absorbent core 10 are not covered with the sheet (e.g. core-wrapping sheets 13, 14), and the absorbent core 10 is exposed on the side surfaces of the absorbent sheet 1. Thus, even when the excreted fluid flows out from the top surface of the absorbent sheet 1, the excreted fluid is absorbed on the side surfaces of the absorbent sheet 1. This can suppress the leakage of the excreted fluid from the absorbent sheet 1. However, the configuration is not limited thereto. The absorbent core 10 need not extend to the side-edge portions 1ab, 1cd, and the side surfaces of the absorbent core 10 need not be exposed but may be covered with the sheet.

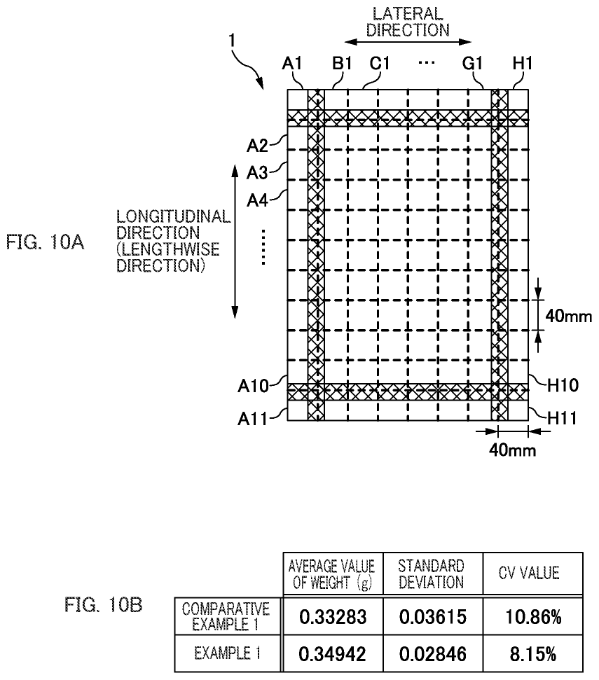

FIG. 10A is a diagram illustrating a method of measuring variation in weight of the absorbent sheet 1, and FIG. 10B is a diagram showing measurement results of variation in weight of the absorbent sheet 1. Regarding variation in weight of the absorbent sheet 1, samples (Example 1, Comparative Example 1) of the absorbent sheet 1 were actually manufactured and measured.

The sample of Example 1 was manufactured using the manufacturing method shown in FIGS. 3 and 4. In Example 1, the planar shape of the sample was a square (rectangle), and the dimension thereof was a longitudinal length of 440 mm×a lateral length of 320 mm. The absorbent core 10 was manufactured using an air laid machine, the basis weight of liquid-absorbent fiber (pulp fiber) was 75 g/m², and the basis weight of superabsorbent polymer was 63 g/m². As shown in FIGS. 2A and 2B, the absorbent core 10 extends to the four side edges 1a to 1d of the absorbent sheet 1 and is exposed on four side surfaces of the absorbent sheet 1. The core-wrapping sheets 13, 14 were tissues (basis weight of 13 g/m²), the top-face sheet 11 was an air-through nonwoven fabric sheet (basis weight of 22 g/m²), and the back-face sheet 12 was a resin film (basis weight of 18 g/m²).

In the sample of Example 1, the absorbent core 10 and the core-wrapping sheet 13, 14 were flat-pressed by press rollers heated to 180° C. Then, as shown in FIG. 4, after all the materials were stacked, water was sprayed onto the materials, and the compressed portions 20 were formed by compression rollers heated to 65° C. As with FIG. 1, the laterally-compressed portions 21 and the longitudinally-compressed portions 22 each had a width of 5 mm, and were formed at positions located inwardly by a length of 7 mm from the side edges la to Id of the absorbent sheet 1. Although the adhesives are not provided between the absorbent core 10 and the core-wrapping sheets 13, 14, the adhesives are provided between the top-face sheet 11 and the core-wrapping sheet 13, and between the back-face sheet 12 and the core-wrapping sheet 14.

The sample of Comparative Example 1 had the same shape and dimension (440 mm×320 mm) as the sample of Example 1. However, unlike the sample of Example 1 (FIG. 1), the laterally-compressed portions 21 and the longitudinally-compressed portions 22 were not formed. The basis weight (75 g/m²) of the liquid-absorbent fiber (pulp fiber) constituting the absorbent core 10 and the basis weight (63 g/m²) of the superabsorbent polymer 102 are also the same as those in Example 1. However, in Comparative Example 1, the absorbent core was manufactured using a pattern drum. Specifically, the absorbent core 10 was constituted by a layer of the superabsorbent polymer formed by the superabsorbent polymer (45 g/m²) being sprayed onto the surface of a part of the absorbent core (liquid-absorbent fibers: 75 g/m², superabsorbent polymer: 18 g/m²) that was manufactured using a mold of the pattern drum. In the sample of Comparative Example 1, the absorbent core 10 was smaller than the absorbent sheet 1, and there was a region where the absorbent core 10 was not present at the outer peripheral edge portions of the absorbent sheet 1 (a width of 25 to 30 mm). The core-wrapping sheets 13, 14 were tissues (top-side basis weight of 13.5 g/m²; back-side basis weight of 13 g/m²), the top-face sheet 11 was an air-through nonwoven fabric sheet (basis weight of 22 g/m²), and the back-face sheet 12 was a resin film (basis weight of 18 g/m²).

In the sample of Comparative Example 1, the core-wrapping sheets 13, 14 and the absorbent core 10 were joined together by adhesives, and the flat pressing was not performed thereto. The top-face sheet 11 and the core-wrapping sheet 13 were joined together by the adhesives, and the core-wrapping sheet 14 and the back-face sheet 12 were also joined together by the adhesives. In Comparative Example 1, the flat pressing and water injection were not performed before formation of the compressed portions 21, 22.

Next, variation in weight of the absorbent sheet of each of Example 1 and Comparative Example 1 was measured. The measurement method is as follows. First, as shown in FIG. 10A, the rectangular plane of the absorbent sheet is divided into square regions having a longitudinal length of 40 mm and a lateral length of 40 mm. Such square regions throughout the absorbent core 10 is present are referred to as “second divided regions”. In each of Example 1 and Comparative Example 1, the absorbent sheet had a longitudinal length of 440 mm and a lateral length of 320 mm. Thus, the absorbent Sheet was divided into eleven regions in the longitudinal direction and into eight regions in the lateral direction. Then, the absorbent sheet was divided into eighty-eight regions (A1 to A11, B1 to B11, . . . H1 to H11).

In the sample of Example 1, the absorbent core 10 extends to the side edges 1a to 1d of the absorbent sheet 1, and thus, all the eighty-eight regions were defined as the second divided region. In the sample of Comparative Example 1, the absorbent core 10 was one size smaller than the absorbent sheet 1. Thus, fifty-four regions (B2 to B10, C2 to C10, . . . G2 to G10) excluding the region on the outer periphery of the absorbent sheet 1 were defined as the second the divided region.

A certain vertex of the absorbent sheet (rectangle) is taken as the origin, and the absorbent sheet is preferably divided into the regions each having the length of 40 mm from the origin toward a longitudinal-one side, and is preferably divided into the regions each having the length of 40 mm from the origin toward a lateral-one side. In the case where each length of sides of the absorbent sheet is not a multiple of 40, the region whose dimension is smaller than 40 mm×40 mm is excluded from the measurement target. In the case where the absorbent sheet does not have a rectangular shape, an imaginary rectangle is formed by one side with the longitudinal maximum length of the absorbent sheet and one side with the lateral maximum length of the absorbent sheet, and then, the absorbent sheet is divided into the regions each having the dimension of 40 mm×40 mm as described above.

After the second divided regions were cut out from the absorbent sheet, the weight (g) of each second divided region was measured. Then, for each sample, the average value (g) of weight of the second divided regions and the standard deviation were calculated, and the CV value was calculated by dividing the standard deviation by the average value (standard deviation/average value). FIG. 10B shows the results.

As shown in FIG. 10B, in the sample of Comparative Example 1, the average weight of fifty-four second divided regions was 0.33283 (g), and the standard deviation was 0.03615. Thus, the CV value was 10.86%. In contrast, in the sample of Example 1, the average weight of eighty-eight second divided regions was 0.34942 (g) and the standard deviation was 0.02846. Thus, the CV value was 8.15%.

The above-mentioned results showed that the absorbent sheet 1 of Example 1 including the absorbent core 10 that was manufactured using the air laid machine had a smaller CV value, which indicates variation, and thus had smaller variation in weight, as compared with the absorbent sheet of Comparative Example 1 including the absorbent core 10 that was manufactured using the pattern drum.

Then, the absorbent sheet 1 was divided into regions each having a longitudinal length of 40 mm and a lateral length of 40 mm, and the regions throughout which the absorbent core 10 is present are defined as the second divided regions (A1 to H11). In this case, it is preferable that the CV value indicating the variation in weight of the second divided regions is 10% or less.

The small variation in weight of the absorbent sheet 1 means that variation in weight of the absorbent core 10 is also small. Thus, as compared with the case where the CV value of weights of the second divided regions is greater than 10%, the force is likely to be uniformly applied to the materials in the thickness direction at a time of flat pressing or formation of the compressed portions 20. As a result, the materials are more firmly bonded to each other in the thickness direction. This can increase the rigidity of the absorbent sheet 1 to suppress the twisting of the absorbent sheet 1. In addition, the folding of the sheet and occurrence of winkles due to weight difference can be suppressed. Therefore, the absorption area of the absorbent sheet 1 is ensured to suppress the leakage of the excreted fluid from the absorbent sheet 1. In addition, since the variation in weight of the absorbent core 10 is small, the excreted liquid can be absorbed and retained regardless of the position of the absorbent sheet 1. This can suppress the leakage from the absorbent sheet 1.

### Other Embodiments

Although the embodiment of the present disclosure has been described hereinabove, the above embodiment of the present disclosure is simply to facilitate understanding of the present disclosure and is not in any way to be construed as limiting the present disclosure. The present disclosure may variously be changed or altered without departing from its gist and encompass equivalents thereof.

For example, regardless of the maximum thickness of the absorbent sheet 1 and the bending rigidity of the absorbent sheet, when the drop test using the artificial urine is performed, the configuration of the absorbent sheet 1 is acceptable in which the CV value indicating variation in the value (bulge value) obtained by subtracting the pre-absorption thickness from the post-absorption thickness in each diffusion divided region is 30% or less; and the maximum value (bulge value) obtained by subtracting the pre-absorption thickness from the post-absorption thickness in each diffusion divided region is 1 mm more. The configuration of such an absorbent sheet 1 is acceptable in which the maximum thickness is greater than 2 mm, and in which the average value of the bending rigidity B of the absorbent sheet in the longitudinal direction and the lateral direction according to the KES method is less than 0.5 gf·cm²/cm. Also in this case, in the above-mentioned absorbent sheet 1, variation in the bulge value can be smaller as compared with the case where the CV value of the bulge value is greater than 30%. This can reduce unevenness on the sheet surface after absorption. Thus, the twisting of the absorbent sheet 1 can be suppressed to ensure the absorption area. As compared with the case where the maximum value of the CV value of the bulge value is less than 1 mm, the absorbent sheet 1 bulges by reliably absorbing and retaining the excreted fluid, thereby suppressing the leakage of the excreted fluid from the absorbent sheet 1.

### Reference Signs List

1: Absorbent Sheet; 2: Absorbent Body;
10: Absorbent Core;
101: Liquid-absorbent Fiber; 102: Superabsorbent Polymer;
11: Top-face sheet; 12: Back-face sheet;
13, 14: Core-wrapping Sheet;
20: Compressed Portion; 21: Laterally-compressed Portion;
22: Longitudinally-compressed Portion;
40: Manufacturing Apparatus; 41, 43: Supply Roller; 42: Air Laid Machine;
44: Water Spray; 45: Press Roller; 46: Wind-up Roller
50; Manufacturing Apparatus; 51, 53, 55: Supply Roller;
52, 54: Adhesive Discharge Portion; 56: Water Spray;
57, 58: Compression Roller; 59: Cut Roller.

1. An absorbent sheet having a longitudinal direction, a lateral direction, and
a thickness direction that are orthogonal to each other, the absorbent sheet comprising:
an absorbent core having liquid-absorbent fiber and superabsorbent polymer;
a top-face sheet disposed on a top-face side in the thickness direction with respect to the absorbent core,
the top-face sheet being liquid permeable; and
a back-face sheet disposed on a back-face side in the thickness direction with respect to the absorbent core,
a maximum thickness of the absorbent sheet being 2.0 mm or less,
an average value of a bending rigidity B of the absorbent sheet in the longitudinal direction according to a KES method being 0.5 gf·cm2/cm or more,
an average value of a bending rigidity B of the absorbent sheet in the lateral direction according to the KES method being 0.5 gf·cm2/cm or more.

2. The absorbent sheet according to claim 1, wherein
the absorbent sheet is divided into four portions in the longitudinal direction and into four portions in the lateral direction,
the divided portions in the absorbent sheet are defined as divided regions,
in the absorbent sheet subjected to a drop test,
the drop test being performed by dropping artificial urine at a predetermined position on the absorbent sheet,
the divided regions where the artificial urine diffuses are defined as diffusion divided regions,
the absorbent sheet before the drop test has a pre-absorption thickness that is a thickness of a portion of each of the diffusion divided regions where the absorbent core is present,
the absorbent sheet after the drop test has a post-absorption thickness that is a thickness of a portion of each of the diffusion divided regions where the absorbent core is present, and
a CV value that indicates variation in a value obtained by subtracting the pre-absorption thickness from the post-absorption thickness in each of the diffusion divided regions, is 30% or less.

3. The absorbent sheet according to claim 2, wherein
a maximum value obtained by subtracting the pre-absorption thickness from the post-absorption thickness in each of the diffusion divided regions is 1 mm or more.

4. An absorbent sheet having a longitudinal direction, a lateral direction, and
a thickness direction that are orthogonal to each other, the absorbent sheet comprising:
an absorbent core having liquid-absorbent fiber and superabsorbent polymer;
a top-face sheet disposed on a top-face side in the thickness direction with respect to the absorbent core,
the top-face sheet being liquid permeable; and
a back-face sheet disposed on a back-face side in the thickness direction with respect to the absorbent core,
the absorbent sheet being divided into four portions in the longitudinal direction and into four portions in the lateral direction,
the divided portions in the absorbent sheet being defined as divided regions, in the absorbent sheet subjected to a drop test,
the drop test being performed by dropping artificial urine at a predetermined position on the absorbent sheet,
the divided regions where the artificial urine diffuses being defined as diffusion divided regions,
the absorbent sheet before the drop test having a pre-absorption thickness that is a thickness of a portion of each of the diffusion divided regions where the absorbent core is present,
the absorbent sheet after the drop test having a post-absorption thickness that is a thickness of a portion of each of the diffusion divided regions where the absorbent core is present,
a CV value that indicates variation in a value obtained by subtracting the pre-absorption thickness from the post-absorption thickness in each of the diffusion divided regions, being 30% or less,
a maximum value obtained by subtracting the pre-absorption thickness from the post-absorption thickness in each of the diffusion divided regions, being 1 mm or more.

5. The absorbent sheet according to claim 2, wherein
a maximum value obtained by subtracting the pre-absorption thickness from the post-absorption thickness in each of the diffusion divided regions, being 2 mm or less.

6. The absorbent sheet according to claim 1, wherein
the absorbent sheet is divided into four portions in the longitudinal direction and into four portions in the lateral direction,
the divided portions in the absorbent sheet being defined as divided regions, and in the absorbent sheet before absorption, a CV value that indicates variation in a thickness of a portion of each of the divided regions where the absorbent core is present is 10% or less.

7. The absorbent sheet according to claim 1, wherein
a core-wrapping sheet is disposed between the absorbent core and at least one of the top-face sheet and the back-face sheet, and
pulp fiber included in the liquid-absorbent fiber is hydrogen-bonded with cellulosic fiber included in the core-wrapping sheet.

8. The absorbent sheet according to claim 1, wherein
a back surface of an absorbent body including the absorbent core is joined onto the back-face sheet by an adhesive,
the adhesive is provided continuously in the longitudinal direction over an entire longitudinal range of the back surface of the absorbent body, and
the adhesive is provided continuously in the lateral direction over an entire lateral range of the back surface of the absorbent body.

9. The absorbent sheet according to claim 1, wherein
in a central portion of the absorbent sheet in the longitudinal direction and the lateral direction,
a compressed portion is not provided in which at least the absorbent core is compressed partially in the planar direction and in the thickness direction.

10. The absorbent sheet according to claim 1, wherein
a density of a portion of the absorbent sheet where the absorbent core is present is 0.35 g/cm3 or less.

11. The absorbent sheet according to claim 1, wherein
the absorbent core extends to a longitudinal side-edge portion of the absorbent sheet, and
the absorbent core extends to a lateral side-edge portion of the absorbent sheet,
the longitudinal side-edge portion being a region extending inwardly from a longitudinal side edge of the absorbent sheet by a distance equal to an average thickness of the absorbent sheet,
the lateral side-edge portion being a region extending inwardly from a lateral side edge of the absorbent sheet by a distance equal to an average thickness of the absorbent sheet.

12. The absorbent sheet according to claim 1, wherein
the absorbent sheet is divided into regions each having a longitudinal length of 40 mm and a lateral length of 40 mm,
the regions throughout which the absorbent core is present are defined as second divided regions, and
a CV value that indicates variation in weight of the second divided regions is 10% or less.
